(19) United States
(12) Patent Application Publication
(10) Pub. No.: US 2019/0167083 A1
(43) Pub. Date: Jun. 6, 2019

(54) ENDOSCOPE SYSTEM

(71) Applicant: OLYMPUS CORPORATION, Tokyo (JP)

(72) Inventor: Toshiaki WATANABE, Tokyo (JP)

(73) Assignee: OLYMPUS CORPORATION, Tokyo (JP)

(21) Appl. No.: 16/272,312

(22) Filed: Feb. 11, 2019

Related U.S. Application Data
(63) Continuation of application No. PCT/JP2017/004497, filed on Feb. 8, 2017.

(30) Foreign Application Priority Data
Sep. 6, 2016 (JP) 2016-173521

(51) Int. Cl. A61B 1/04; A61B 1/05; A61B 1/00; A61B 1/06

(52) U.S. Cl. CPC A61B 1/043; A61B 1/05; G02B 23/2461; A61B 1/06; A61B 1/00096; A61B 1/00163

(57) ABSTRACT

An endoscope system includes: a light source apparatus configured to emit excitation light for exciting a fluorescent medical agent and illuminating light; an image sensor configured to pick up an image of each of fluorescence emitted in response to application of the excitation light to an object present inside a body cavity of a subject to which the fluorescent medical agent is administered and reflected light emitted in response to application of the illuminating light to the object; and a mixed image generating circuit configured to generate an observation image in which a first image that is an image obtained by performing image pickup of the reflected light by the image sensor, and a second image that is an image indicating a site of emission of the fluorescence in the object are mixed in a set of pixel arrays that do not overlap each other and each have periodicity.

### CROSS REFERENCE TO RELATED APPLICATION

This application is a continuation application of PCT/JP2017/004497 filed on Feb. 8, 2017 and claims benefit of Japanese Application No. 2016-173521 filed in Japan on Sep. 6, 2016, the entire contents of which are incorporated herein by this reference.

### BACKGROUND OF THE INVENTION

### 1. Field of the Invention

The present invention relates to an endoscope system and specifically relates to an endoscope system to be used for fluorescence observation.

### 2. Description of the Related Art

In the medical field, for example, fluorescence observation, which is an observation method in which based on a state of fluorescence emitted when excitation light for exciting a fluorescent medical agent administered to a subject is applied to a desired object present inside a body cavity of the subject, whether or not a lesion part is included in the desired object is examined has conventionally been performed. For example, International Publication No. 2005/048826 discloses a sentinel lymph node detection apparatus to be used for fluorescence observation such as stated above.

More specifically, International Publication No. 2005/048826 discloses a configuration in which excitation light is applied to a living body observed part including a lymph node in the vicinity of a tumor with a fluorescent dye injected in advance and fluorescence emitted from the living body observed part in response to the application of the excitation light is subjected to image pickup to obtain a fluorescence image. Also, International Publication No. 2005/048826 discloses a configuration in which an observation image in which a fluorescence image obtained by image pickup of fluorescence emitted from a living body observed part in response to application of excitation light and a normal image obtained by performing image pickup of reflected light of the excitation light applied to the living body observed part are superimposed on each other is acquired and displayed on an image display apparatus with a luminance and/or a contrast of the acquired observation image adjusted.

### SUMMARY OF THE INVENTION

An endoscope system according to an aspect of the present invention includes: a light source apparatus configured to be capable of emitting excitation light for exciting a fluorescent medical agent administered to a subject and illuminating light for illuminating an inside of a body cavity of the subject; an image sensor configured to pick up an image of each of fluorescence emitted in response to application of the excitation light to an object present inside the body cavity of the subject to which the fluorescent medical agent is administered and reflected light emitted in response to application of the illuminating light to the object; and a mixed image generating circuit configured to generate an observation image in which a first image that is an image obtained by performing image pickup of the reflected light by the image sensor, and a second image that is an image indicating a site of emission of the fluorescence in the object are mixed in a set of pixel arrays that do not overlap each other and each have periodicity.

### BRIEF DESCRIPTION OF THE DRAWINGS

FIG. 1 is a diagram illustrating a configuration of a major part of an endoscope system according to an embodiment;

FIG. 2 is a diagram illustrating an example of pixel arrays in an observation image generated by the endoscope system according to the embodiment;

FIG. 3 is a diagram illustrating an example of pixel arrays in an observation image generated by the endoscope system according to the embodiment;

FIG. 4 is a diagram illustrating an example of pixel arrays in an observation image generated by the endoscope system according to the embodiment;

FIG. 5 is a diagram illustrating an example of pixel arrays in an observation image generated by the endoscope system according to the embodiment;

FIG. 6 is a diagram illustrating an example of pixel arrays in an observation image generated by the endoscope system according to the embodiment;

FIG. 7 is a diagram illustrating an example of a configuration where an observation image is generated using a white light image subjected to contour enhancement processing by a contour enhancement processing section;

FIG. 8 is a diagram illustrating an example of a spatial filter that can be used in the contour enhancement processing by the contour enhancement processing section in FIG. 7;

FIG. 9 is a diagram illustrating an example of a configuration where an observation image is directly subjected to contour enhancement processing by a contour enhancement processing section;

FIG. 10 is a diagram illustrating an example of a spatial filter that can be used in the contour enhancement processing by the contour enhancement processing section in FIG. 9;

FIG. 11 is a diagram illustrating a configuration of a major part of an endoscope system according to a first modification of the embodiment;

FIG. 12 is a diagram illustrating a configuration of a major part of an endoscope system according to a second modification of the embodiment; and

FIG. 13 is a diagram illustrating an example of pixel arrays in an observation image generated by the endoscope system according to the second modification of the embodiment.

### DETAILED DESCRIPTION OF THE PREFERRED EMBODIMENTS

An embodiment of the present invention will be described below with reference to the drawings.

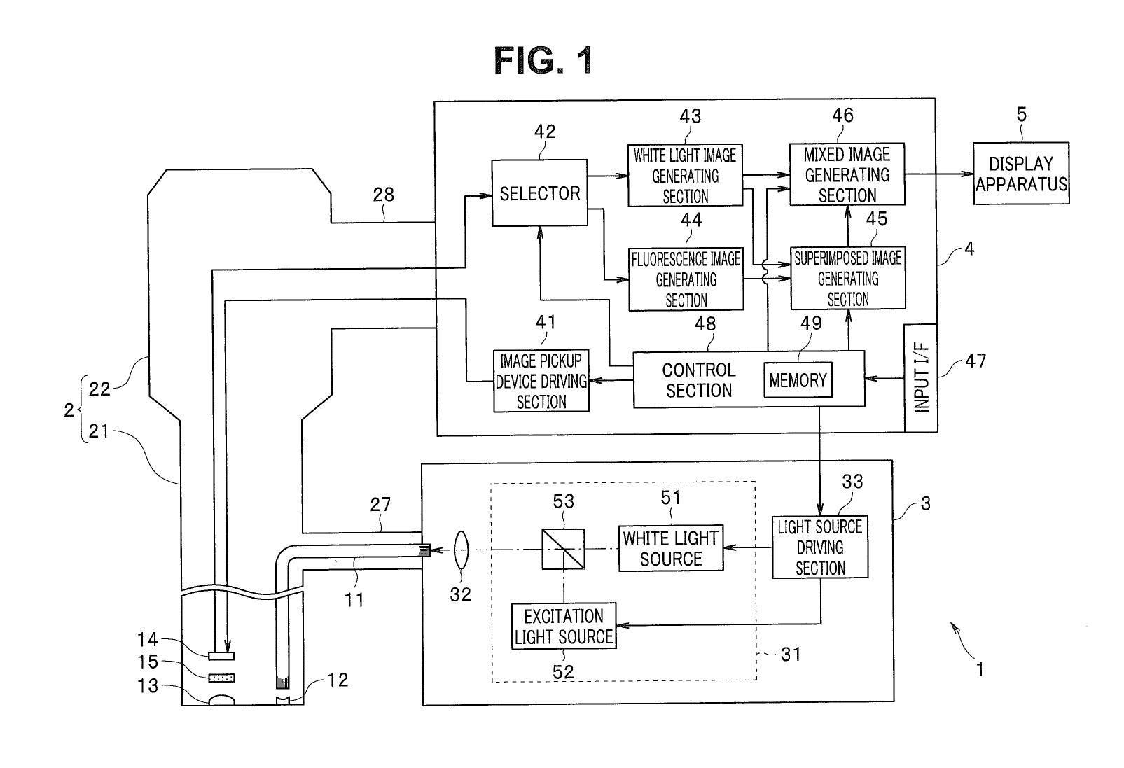

FIGS. 1 to 13 relate to an embodiment of the present invention.

As illustrated in FIG. 1, an endoscope system 1 includes, for example, an endoscope 2 to be inserted into a body cavity of a subject, the endoscope 2 being configured to pick up an image of an object, such as a living body tissue, present inside the body cavity, and output an image pickup signal; a light source apparatus 3 configured to supply light to be applied to the object to the endoscope 2, a processor 4 configured to generate an observation image by subjecting the image pickup signal outputted from the endoscope 2 to various types of processing and output the observation image, and a display apparatus 5 configured to display the observation image outputted from the processor 4 on a screen. FIG. 1 is a diagram illustrating a configuration of a major part of an endoscope system according to the embodiment.

As illustrated in FIG. 1, the endoscope 2 includes, for example, an insertion portion 21 formed in an elongated shape so that the insertion portion 21 can be inserted into a body cavity of a subject, and an operation section 22 provided on the proximal end side of the insertion portion 21. Also, the endoscope 2 is detachably attachable to the light source apparatus 3 via a light guide cable 27. Also, the endoscope 2 is detachably attachable to the processor 4 via a signal cable 28 provided so as to extend from the operation section 22.

A light guide 11 for transmitting light supplied from the light source apparatus 3 is inserted inside the insertion portion 21 and the light guide cable 27.

As illustrated in FIG. 1, an output end portion of the light guide 11 is disposed in the vicinity of an illumination lens 12 in a distal end portion of the insertion portion 21. Also, as illustrated in FIG. 1, an input end portion of the light guide 11 is disposed in the vicinity of a collective lens 32 in the light source apparatus 3 connected to the endoscope 2 via the light guide cable 27.

The illumination lens 12 for outputting light transmitted by the light guide 11 to the outside and an objective lens 13 for receiving light entering from the outside are provided in the distal end portion of the insertion portion 21. Also, an image pickup device 14 and an excitation light cut filter 15 disposed on an optical path from the objective lens 13 to the image pickup device 14 are provided in the distal end portion of the insertion portion 21.

The image pickup device 14 includes, for example, a color CMOS image sensor that includes a primary color filter or a complimentary color filter attached to an image pickup surface and is configured to perform image pickup operation in response to an image pickup device driving signal outputted from the processor 4. Also, the image pickup device 14 is configured so as to pick up an image of light having passed through the excitation light cut filter 15, generate an image pickup signal and output the generated image pickup signal to the processor 4.

The excitation light cut filter 15 has, for example, an optical characteristic of blocking a wavelength band that is the same as a wavelength band of excitation light EXA (which will be described later) outputted via the objective lens 13 and transmitting wavelength bands that are different from the wavelength band of the excitation light EXA, from among respective wavelength bands of light. In other words, the excitation light cut filter 15 has an optical characteristic of transmitting fluorescence FLA (which will be described later) emitted from a fluorescent medical agent in response to application of excitation light EXA.

In other words, an image pickup section in the present embodiment includes the image pickup device 14 and the excitation light cut filter 15.

The operation section 22 is provided on the proximal end side of the insertion portion 21 and has a shape that enables the operation section 22 to be grasped by a user such as a surgeon. Also, at the operation section 22, for example, one or more scope switches (not illustrated), which are switches enabling provision of various instructions to the processor 4 according to operation by the user, are provided.

As illustrated in FIG. 1, the light source apparatus 3 includes, for example, a light emitting section 31, the collective lens 32 and a light source driving section 33.

The light emitting section 31 includes a white light source 51, an excitation light source 52 and a dichroic mirror 53.

The white light source 51 includes, for example, a xenon lamp, a white LED or LEDs of three colors (red, green and blue). Also, the white light source 51 is configured to emit white light WLA, which is, for example, light including respective wavelength bands in a red range, a green range and a blue range, in response to a light source driving signal outputted from the light source driving section 33. Here, in the present embodiment, instead of the white light source 51, for example, a broadband light source including a lamp configured to emit broadband light at least having a wavelength band in a range from the blue range to a near-infrared range, and an optical filter having an optical characteristic of transmitting a wavelength band that is the same as a wavelength band of white light WLA and blocking the other wavelength bands, from among the respective wavelength bands included in the broadband light, may be provided in the light source apparatus 3.

The excitation light source 52 includes, for example, an LD (laser diode). Also, the excitation light source 52 is configured to emit, for example, excitation light EXA, which is a narrowband light including an excitation wavelength for a predetermined fluorescent medical agent administered to a subject, in response to a light source driving signal outputted from the light source driving section 33. In the below, the description will be given assuming that: a fluorescent medical agent administered to a subject is ICG (indocyanine green); excitation light EXA is narrowband near-infrared light including an excitation wavelength for ICG; and fluorescence FLA, which is near-infrared light belonging to a wavelength band on the long wavelength side relative to the excitation light EXA, is emitted from the ICG unless otherwise stated.

The dichroic mirror 53 has, for example, an optical characteristic of transmitting white light WLA emitted from the white light source 51 and outputting the white light WLA to the collective lens 32 side and reflecting excitation light EXA emitted from the excitation light source 52 and outputting the excitation light EXA to the collective lens 32 side.

In other words, the light emitting section 31 is configured to be capable of emitting white light WLA by causing the white light source 51 to emit light in response to a driving signal outputted from the light source driving section 33. Also, the light emitting section 31 is configured to be capable of emitting excitation light EXA by causing the excitation light source 52 to emit light in response to a driving signal outputted from the light source driving section 33. Also, the light emitting section 31 is configured to be capable of outputting the white light WLA and the excitation light EXA to the collective lens 32.

The collective lens 32 is configured to collect light outputted from the light emitting section 31 and outputs the light to the input end portion of the light guide 11.

The light source driving section 33 is configured to generate a light source driving signal for driving the white light source 51 and the excitation light source 52 based on a control signal outputted from the processor 4 and output the light source driving signal to the light emitting section 31.

In other words, the light source apparatus 3 is configured to be capable of emitting excitation light EXA for exciting a fluorescent medical agent administered to a subject and white light WLA, which is illuminating light for illuminating the inside of a body cavity of the subject.

As illustrated in FIG. 1, the processor 4 includes, for example, an image pickup device driving section 41, a selector 42, a white light image generating section 43, a fluorescence image generating section 44, a superimposed image generating section 45, a mixed image generating section 46, an input I/F (interface) 47 and a control section 48. Note that according to the present embodiment, for example, each of the sections of the processor 4 may be configured as an individual electronic circuit or may be configured as a circuit block in an integrated circuit such as an FPGA (field-programmable gate array).

The image pickup device driving section 41 is configured to generate an image pickup device driving signal for driving the image pickup device 14, based on a control signal outputted from the control section 48 and output the image pickup device driving signal.

The selector 42 is configured to perform operation for setting an output destination of an image pickup signal outputted from the endoscope 2 to either the white light image generating section 43 or the fluorescence image generating section 44, based on a control signal outputted from the control section 48.

The white light image generating section 43 is configured to generate a white light image WIA based on an image pickup signal outputted via the selector 42 and output the generated white light image WIA to each of the superimposed image generating section 45 and the mixed image generating section 46. In other words, the white light image generating section 43 is configured to generate a white light image WIA, which is an image obtained by performing image pickup of reflected light of white light WLA by the image pickup device 14.

The fluorescence image generating section 44 is configured to generate a fluorescence image FIA based on an image pickup signal outputted via the selector 42 and output the generated fluorescence image FIA to the superimposed image generating section 45. In other words, the fluorescence image generating section 44 is configured to generate a fluorescence image FIA, which is an image obtained by performing image pickup of fluorescence FLA by the image pickup device 14.

The superimposed image generating section 45 is configured to be capable of performing operation according to a control signal outputted from the control section 48. Also, the superimposed image generating section 45 is configured to generate a superimposed image SIA by performing processing for superimposing a white light image WIA outputted from the white light image generating section 43 and a fluorescence image FIA outputted from the fluorescence image generating section 44 on each other and output the generated superimposed image SIA to the mixed image generating section 46.

More specifically, the superimposed image generating section 45 performs, for example, processing for superimposing a pixel value of a pixel WP at one pixel position in a white light image WIA outputted from the white light image generating section 43 and a pixel value of a pixel FP at the one pixel position in the fluorescence image FIA outputted from the fluorescence image generating section 44 on each other using equation (1) below to obtain a pixel value of a pixel SP at the one pixel position in a superimposed image SIA, for each of all areas of the image.

In equation (1) below, Ri represents a luminance value of a red component in a pixel WP, Gi represents a luminance value of a green component in the pixel WP, Bi represents a luminance value of a blue component in the pixel WP, Fi represents a luminance value (of a fluorescence component) of a pixel FP, Ro represents a luminance value of a red component of a pixel SP, Go represents a luminance value of a green component of the pixel SP, and Bo represents a luminance value of a blue component of the pixel SP. Also, each of α, β and γ in equation (1) below represents a weight coefficient for prescribing a color tone of a site of emission of fluorescence FLA included in a superimposed image SIA, and for example, may be a fixed value set in advance by the superimposed image generating section 45 or a variable value set according to a control signal from the control section 48.

$$( \begin{matrix}R o \\ G o \\ B o\end{matrix} ) = ( \begin{matrix}1 & 0 & 0 & α \\ 0 & 1 & 0 & β \\ 0 & 0 & 1 & γ\end{matrix} ) ( \begin{matrix}R i \\ G i \\ B i \\ F i\end{matrix} ) \qquad ( 1 )$$

In other words, superimposed image generating section 45 is configured to generate a superimposed image SIA, which is an image indicating a site of emission of fluorescence FLA in an object subjected to image pickup by the endoscope 2.

The mixed image generating section 46 is configured to generate an observation image by performing processing for mixing a part of a white light image WIA outputted from the white light image generating section 43 and a part of a superimposed image SIA outputted from the superimposed image generating section 45, based on a control signal outputted from the control section 48 and output the generated observation image to the display apparatus 5. Details of the processing performed in the mixed image generating section 46 will be described later.

The input I/F 47 includes one or more switches and/or buttons each enabling provision of an instruction according to operation by a user.

The control section 48 is configured to be capable of generating control signals for causing operation according to an instruction from the input I/F 47 to be performed and outputting the control signals to the light source driving section 33, the superimposed image generating section 45 and the mixed image generating section 46, respectively. Also, the control section 48 includes a memory 49 in which control information to be used for controlling respective sections of the endoscope system 1 is stored.

The control section 48 is configured to generate control signals for synchronizing a timing of emission of white light WLA and excitation light EXA in the light emitting section 31, image pickup operation in the image pickup device 14 and a destination of an image pickup signal inputted to the processor 4 with one another and output the control signals to the light source driving section 33, the image pickup device driving section 41 and the selector 42, respectively.

The control section 48 is configured to generate a control signal for extracting pixels WP included in a white light image WIA and pixels SP included in a superimposed image SIA according to a set of pixel arrays that do not overlap each other and each have periodicity, based on control information read from the memory 49 and output the control signal to the mixed image generating section 46.

Next, operation, etc., of the endoscope system 1 according to the present embodiment will be described. In the below, the description will be given assuming that ICG (fluorescent medical agent) is administered in advance to a desired object present inside a body cavity of a subject before fluorescence observation of the desired object is performed. Also, in the below, for simplicity, description of white light observation, which is an observation method in which a white light image WIA obtained by picking up an image of an object to which white light WLA is applied is displayed as an observation image on the display apparatus 5, will be omitted.

First, after connecting the respective sections of the endoscope system 1 and turning on the power, a user provides, for example, an instruction for starting a fluorescence observation of an object to the control section 48 by operating a fluorescence observation start switch (not illustrated) in the input I/F 47. Also, the user disposes the distal end portion of the insertion portion 21 in the vicinity of a desired object present inside a body cavity of a subject by inserting the insertion portion 21 into the body cavity.

When the processor 4 is powered up, the control section 48 reads control information from the memory 49 and generates a control signal for extracting pixels WP included in a white light image WIA and pixels SP included in a superimposed image SIA according to a set of pixel arrays that do not overlap each other and each have periodicity, based on the read control information and outputs the control signal to the mixed image generating section 46.

More specifically, for example, the control section 48 generates a control signal for extracting a pixel group WGA including a plurality of pixels WP present at positions in a first pixel array of the set of pixel arrays that do not overlap each other and each have periodicity, from among the respective pixels included in the white light image WIA, and extracting a pixel group SGA including a plurality of pixels SP present at positions in a second pixel array of the set of pixel arrays, from among the respective pixels included in the superimposed image SIA, and outputs the control signal to the mixed image generating section 46.

When the processor 4 is powered up and the control section 48 detects the instruction from the fluorescence observation start switch in the input I/F 47, the control section 48 generates control signals for synchronizing a timing of emission of white light WLA and excitation light EXA in the light emitting section 31, image pickup operation in the image pickup device 14 and an output destination of an image pickup signal inputted to the processor 4 with one another and outputs the control signals to the light source driving section 33, the image pickup device driving section 41 and the selector 42, respectively.

More specifically, for example, the control section 48 generates a control signal for causing the image pickup device 14 to perform rolling shutter-type image pickup operation and outputs the control signal to the image pickup device driving section 41. Also, for example, the control section 48 generates a control signal for causing white light WLA in a light amount AL1 and excitation light EXA in the light amount AL1 to be emitted alternately (in a time-division manner) in blanking periods in which no reading is performed for all of lines in the image pickup device 14 in rolling shutter-type image pickup operation, and outputs the control signal to the light source driving section 33. Also, for example, the control section 48 generates a control signal for setting an output destination of an image pickup signal to be inputted to the processor 4 at the time of emission of white light WLA to the white light image generating section 43 and setting an output destination of an image pickup signal to be inputted to the processor 4 at the time of emission of excitation light EXA to the fluorescence image generating section 44 and outputs the control signal to the selector 42.

According to the above-described control by the control section 48, for example, in a first blanking period in the image pickup device 14, white light WLA is applied to an object, reflected light of the white light WLA, which is return light emitted from the object, is subjected to image pickup by the image pickup device 14, an image pickup signal generated by the image pickup device 14 is outputted to the white light image generating section 43 via the selector 42, and a white light image WIA generated based on the image pickup signal is outputted to each of the superimposed image generating section 45 and the mixed image generating section 46.

Also, according to the above-described control by the control section 48, for example, in a second blanking period in the image pickup device 14, the second blanking period being different from the aforementioned first blanking period, excitation light EXA is applied to the object, fluorescence FLA contained in return light emitted from the object is subjected to image pickup by the image pickup device 14, an image pickup signal generated by the image pickup device 14 is outputted to the fluorescence image generating section 44 via the selector 42, a fluorescence image FIA generated based on the image pickup signal is outputted to the superimposed image generating section 45.

The superimposed image generating section 45 generates a superimposed image SIA by, for example, superimposing the white light image WIA outputted from the white light image generating section 43 and the fluorescence image FIA outputted from the fluorescence image generating section 44 on each other in a state in which coefficients α, β and γ in equation (1) above are set as α=γ=0 and β=1 and outputs the generated superimposed image SIA to the mixed image generating section 46. In other words, according to such operation of the superimposed image generating section 45 as above, the superimposed image SIA in which a site of emission of the fluorescence FLA in the object subjected to image pickup by the endoscope 2 is indicated in green is outputted to the mixed image generating section 46.

The mixed image generating section 46 generates an observation image by performing processing for mixing a part of the white light image WIA outputted from the white light image generating section 43 and a part of the superimposed image SIA outputted from the superimposed image generating section 45 based on the control signal outputted from the control section 48, and outputs the generated observation image to the display apparatus 5.

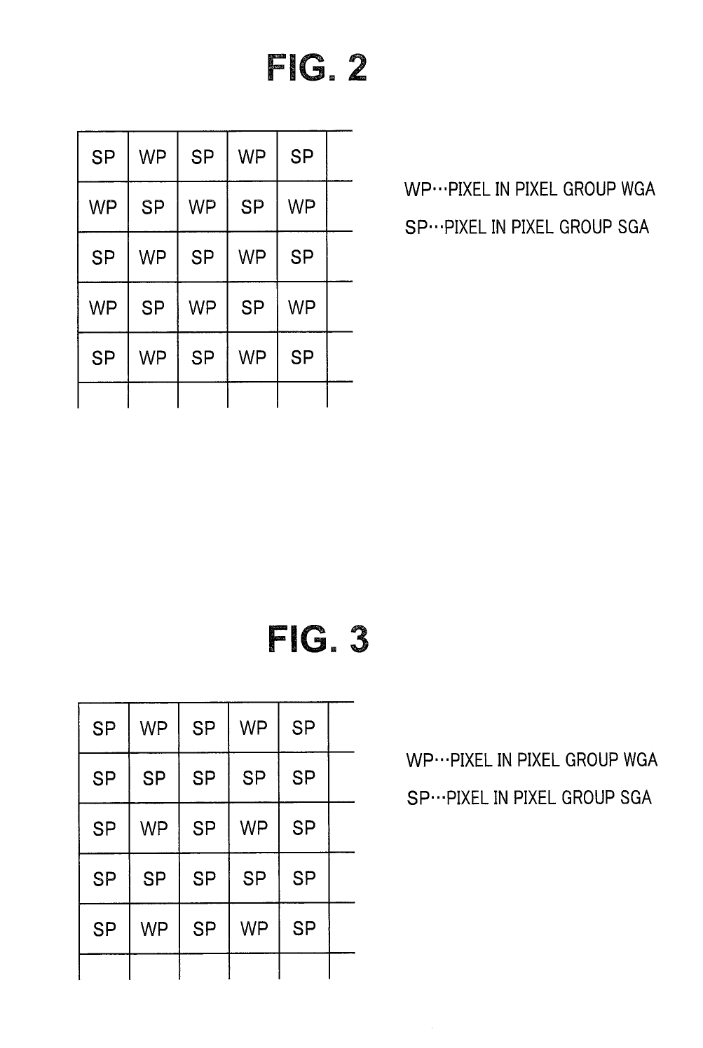

More specifically, the mixed image generating section 46 generates an observation image by, for example, extracting the pixel group WGA according to a first checkered pixel array and extracting the pixel group SGA according to a second checkered pixel array that does not overlap the first checkered pixel array, based on the control signal outputted from the control section 48 and mixing the extracted pixel groups WGA and SGA, and outputs the generated observation image to the display apparatus 5. As a result of such operation above being performed by the mixed image generating section 46, for example, as illustrated in FIG. 2, an observation image in which pixels WP and pixels SP are alternately disposed in a one pixel-by-one pixel basis and the number of pixels WP and the number of pixels SP are equal to each other is generated. Also, where such operation as above is performed in the mixed image generating section 46, information for extracting the pixel group WGA according to the first checkered pixel array and extracting the pixel group SGA according to the second checkered pixel array that does not overlap the first checkered pixel array is included in the control information stored in the memory 49. FIG. 2 is a diagram illustrating an example of pixel arrays in an observation image generated by the endoscope system according to the embodiment.

As described above, the endoscope system 1 according to the present embodiment enables displaying an observation image in which information indicating a site of emission of fluorescence FLA included in a superimposed image SIA is added while information indicating a structure, including, e.g., projections and recesses, of a living body tissue included in a white light image WIA is left as much as possible, on the display apparatus 5. Therefore, the present embodiment enables when fluorescence emitted from a living body tissue is observed, easily grasping a structure of the living body tissue at a site of emission of the fluorescence in comparison with the conventional techniques.

Also, according to the present embodiment, the configuration of the endoscope system 1 may arbitrarily be modified so that a fluorescent medical agent other than ICG is available.

More specifically, for example, where a fluorescent medical agent administered to a subject is fluorescein, it is possible that: narrowband blue light including an excitation wavelength for fluorescein is emitted as excitation light EXB from the excitation light source 52; a half mirror configured to transmit white light WLA and reflect excitation light EXB is provided instead of the dichroic mirror 53; light having a wavelength band that is the same as a wavelength band of the excitation light EXB is blocked by the excitation light cut filter 15; and light in a visible light range, the light including fluorescence FLB, which is green light emitted from the fluorescein in response to application of the excitation light EXB, is caused to pass through the excitation light cut filter 15.

Also, according to the present embodiment, information for extracting pixel groups WGA and SGA along pixel arrays other than the aforementioned checkered pixel arrays may be included in the control information stored in the memory 49 as long as such pixel arrays is a set of pixel arrays that do not overlap each other and each have periodicity.

More specifically, according to the present embodiment, for example, information for extracting a pixel WP present on an upper right part of each of small areas of 2×2 pixels obtained as a result of a white light image WIA being divided into the small area, as a pixel belonging to a pixel group WGA, and extracting respective pixels SP present in parts other than an upper right part of each of small areas of 2×2 pixels obtained as a result of a superimposed image SIA being divided into the small areas, as pixels belonging to a pixel group SGA, may be included in the control information stored in the memory 49. Then, where the mixed image generating section 46 extracts pixel groups WGA and SGA according to such set of pixel arrays as above, for example, as illustrated in FIG. 3, an observation image in which pixels SP and pixels WP are disposed alternately on each of odd-numbered horizontal lines, only pixels SP are disposed on each of even-numbered horizontal lines and the number of pixels SP is triple of the number of pixels WP is generated. FIG. 3 is a diagram illustrating an example of pixel arrays in an observation image generated by the endoscope system according to the embodiment.

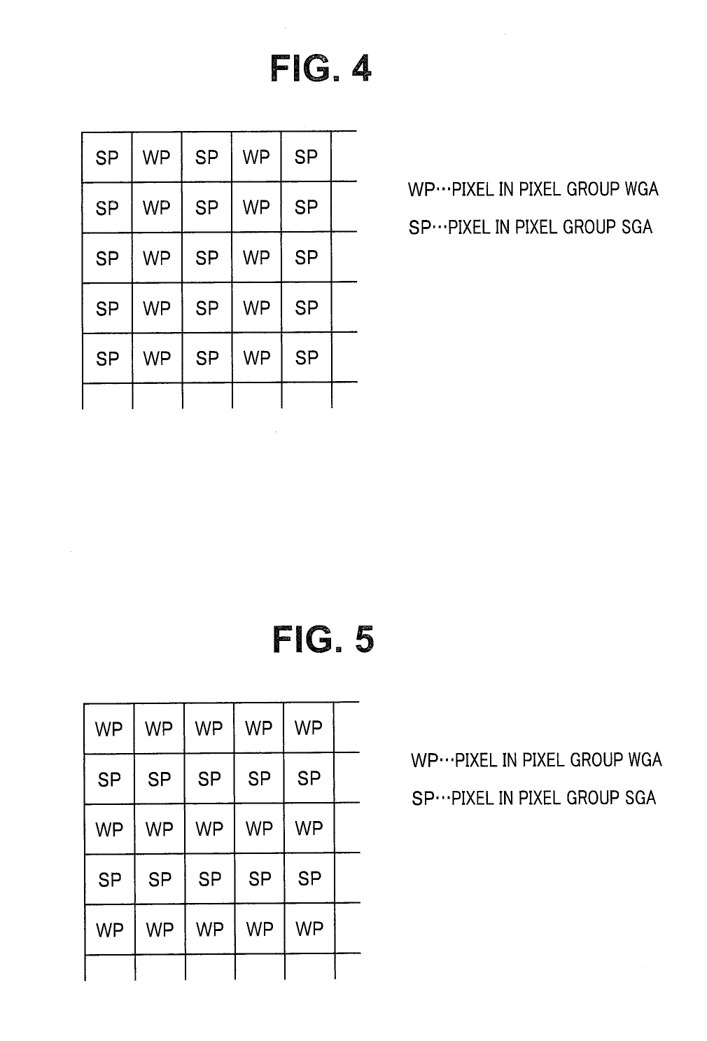

Also, according to the present embodiment, for example, information for extracting a pixel group WGA according to a first vertically striped pixel array and extracting a pixel group SGA along a second vertically striped pixel array that does not overlap the first vertically striped pixel array may be included in the control information stored in the memory 49. Then, where the mixed image generating section 46 extracts pixel groups WGA and SGA along such set of pixel arrays as above, for example, as illustrated in FIG. 4, an observation image in which only pixels SP are disposed on each of odd-numbered vertical lines, only pixels WP are disposed on each of even-numbered vertical lines and the number of pixels WP and the number of pixels SP are equal to each other is generated. FIG. 4 is a diagram illustrating an example of pixel arrays in an observation image generated by the endoscope system according to the embodiment.

Also, according to the present embodiment, for example, information for extracting a pixel group WGA according to a first horizontally striped pixel array and extracting a pixel group SGA according to a second horizontally striped pixel array that does not overlap the first horizontally striped pixel array may be included in the control information stored in the memory 49. Then, where the mixed image generating section 46 extracts pixel groups WGA and SGA according to such set of pixel arrays as above, for example, as illustrated in FIG. 5, an observation image in which only pixels WP are disposed on each of odd-numbered horizontal line, only pixels SP are disposed on each of even-numbered horizontal lines and the number of pixels WP and the number of pixels SP are equal to each other is generated. FIG. 5 is a diagram illustrating an example of pixel arrays included in an observation image generated by the endoscope system according to the embodiment.

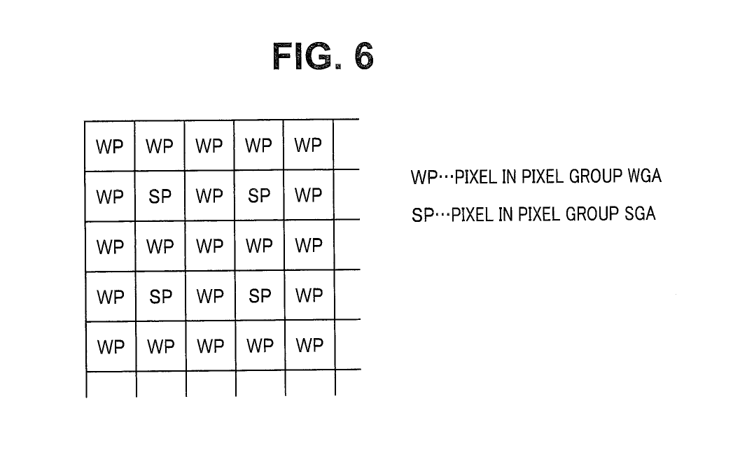

Also, according to the present embodiment, for example, information for extracting a pixel SP present in a lower right part of each of small areas of 2×2 pixels obtained as a result of a superimposed image SIA being divided into the small areas, as a pixel belonging to a pixel group SGA, and extracting respective pixels WP present in parts other than a lower right part of each of small areas of 2×2 pixels obtained as a result of a white light image WIA being divided into the small areas, as pixels belonging to a pixel group WGA, may be included in the control information stored in the memory 49. Then, where the mixed image generating section 46 extracts pixel groups WGA and SGA according to such set of pixel arrays as above, for example, as illustrated in FIG. 6, an observation image in which only pixels WP are disposed on each of odd-numbered horizontal lines, pixels WP and pixels SP are alternately disposed on each of even-numbered horizontal lines and the number of pixels WP is triple the number of pixels SP is generated. FIG. 6 is a diagram illustrating an example of pixel arrays in an observation image generated by the endoscope system according to the embodiment.

Also, according to the present embodiment, for example, a configuration in which where information indicating two or more sets of pixel arrays to be used for generation of an observation image by the mixed image generating section 46 is included in the control information stored in the memory 49, an instruction for displaying a desired observation image according to a set of pixel arrays from among the two or more sets of pixel arrays is provided via the input I/F 47 and a control signal for extracting pixel groups WGA and SGA according to the set of pixel arrays is outputted from the control section 48 to the mixed image generating section 46 may be employed.

Then, such configuration as above enables, for example, when an instruction for displaying an observation image with emphasis placed more on visibility of a site of emission of fluorescence FLA than on visibility of a structure of a living body tissue is provided via the input IF 47, generating an observation image including pixel arrays such as illustrated in FIG. 3 and displaying the observation image on the display apparatus 5. Also, such configuration as above enables, for example, when an instruction for displaying an observation image with emphasis more on visibility of a structure of a living body tissue than on visibility of a site of emission of fluorescence FLA is provided via the input I/F 47, generating an observation image including pixel arrays such as illustrated in FIG. 6 and displaying the observation image on the display apparatus 5. Also, such configuration as above enables when an instruction for displaying an observation image with emphasis placed on a balance in visibility between a structure of a living body tissue and a site of emission of fluorescence FLA is provided via the input I/F 47, generating an observation image including pixel arrays such as illustrated in FIG. 2, FIG. 4 or FIG. 5 and displaying the observation image on the display apparatus 5.

Also, according to the present embodiment, for example, the control section 48 may be configured to when the control section 48 outputs a control signal for extracting pixel groups WGA and SGA in such a manner that the number of pixels WP and the number of pixels SP are different from each other to the mixed image generating section 46, output a control signal for setting a value of β in equation (1) above to a value according to the difference in number of pixels to the superimposed image generating section 45.

More specifically, the control section 48 may be configured to, for example, when the control section 48 outputs a control signal for extracting pixel groups WGA and SGA in such a manner that the number of pixels SP is triple the number of pixels WP to the mixed image generating section 46, output a control signal for setting the value of β in equation (1) above to ⅓ to the superimposed image generating section 45. Then, according to such control as above by the control section 48, when the superimposed image generating section 45 generates a superimposed image SIA using equation (1) above, weight coefficient β (=⅓) applied to the fluorescence image FIA is set to be ⅓ a weight coefficient (=1) applied to a white light image WIA.

Also, the control section 48 may be configured to, for example, when the control section 48 outputs a control signal for extracting pixel groups WGA and SGA in such a manner that the number of pixels WP is triple the number of pixels SP to the mixed image generating section 46, output a control signal for setting the value of β in equation (1) above to 3 to the superimposed image generating section 45. Then, according to such control as above by the control section 48, when the superimposed image generating section 45 generates a superimposed image SIA using equation (1) above, weight coefficient β (=3) applied to a fluorescence image FIA is set to be triple a weight coefficient (=1) applied to a white light image WIA.

Also, according to the present embodiment, for example, the control section 48 may be configured to, when the control section 48 outputs a control signal for extracting pixel groups WGA and SGA in such a manner that the number of pixels WP and the number of pixels SP are different from each other to the mixed image generating section 46, output a control signal for setting an amount of white light WLA and an amount of excitation light EXA to respective amounts according to the difference in pixel number to the light source driving section 33.

More specifically, the control section 48 may be configured to, for example, when the control section 48 outputs a control signal for extracting pixel groups WGA and SGA in such a manner that the number of pixels SP is triple the number of pixels WP to the mixed image generating section 46, output a control signal for causing an amount of white light WLA to be triple an amount of excitation light EXA to the light source driving section 33. Then, according to such control as above by the control section 48, the amount of excitation light EXA emitted from the light emitting section 31 is set to a light amount AL2, and the amount of white light WLA emitted from the light emitting section 31 is set to a light amount AL3, which is triple the light amount AL2.

Also, the control section 48 may be configured to, for example, when the control section 48 outputs a control signal for extracting pixel groups WGA and SGA in such a manner that the number of pixels WP is triple the number of pixels SP to the mixed image generating section 46, output a control signal for causing an amount of excitation light EXA to be triple an amount of white light WLA to the light source driving section 33. Then, according to such control as above by the control section 48, an amount of white light WLA emitted from the light emitting section 31 is set to a light amount AL4 and an amount of excitation light EXA emitted from the light emitting section 31 is set to a light amount AL5, which is triple the light amount AL4.

Also, according to the present embodiment, for example, a set of pixel arrays to be used for extraction of pixel groups WGA and SGA may be determined according to a type of surgical operation performed for a subject and/or a fluorescent medical agent administered to the subject.

More specifically, for example, a configuration in which where information indicating a plurality of initial settings in which a type of surgical operation performed for a subject and/or a type of fluorescent medical agent administered to the subject, and pixel arrays to be used for extraction of pixel groups WGA and SGA are associated with each other is included in the control information stored in the memory 49, an instruction for selecting a type of surgical operation and/or a type of fluorescent medical agent is provided via the input I/F 47 and a control signal for extracting pixel groups WGA and SGA using a set of pixel arrays included in an initial setting corresponding to the instruction is outputted from the control section 48 to the mixed image generating section 46 may be employed. Then, according to such configuration above, for example, when an instruction for selecting ICG as a fluorescent medical agent is provided via the input I/F 47, a control signal for extracting a pixel group WGA according to a first checkered pixel array and extracting a pixel group SGA according to a second checkered pixel array that does not overlap the first checkered pixel array based on an initial setting corresponding to the instruction is outputted from the control section 48 to the mixed image generating section 46.

Also, according to the present embodiment, for example, a configuration in which the mixed image generating section 46 includes the functions of the superimposed image generating section 45 may be employed.

More specifically, based on a control outputted from the control section 48, for example, the mixed image generating section 46 can generate a pixel group WGA by setting each of coefficients α, β and γ in equation (1) above to 0 for pixels according to a first checkered pixel array and can generate a pixel group SGA by setting α=γ=0 and β=1 for pixels according to a second checkered pixel array that does not overlap the first checkered pixel array. An observation image in which the pixel groups WGA and SGA are mixed is generated in this way and the generated observation image is outputted to the display apparatus 5.

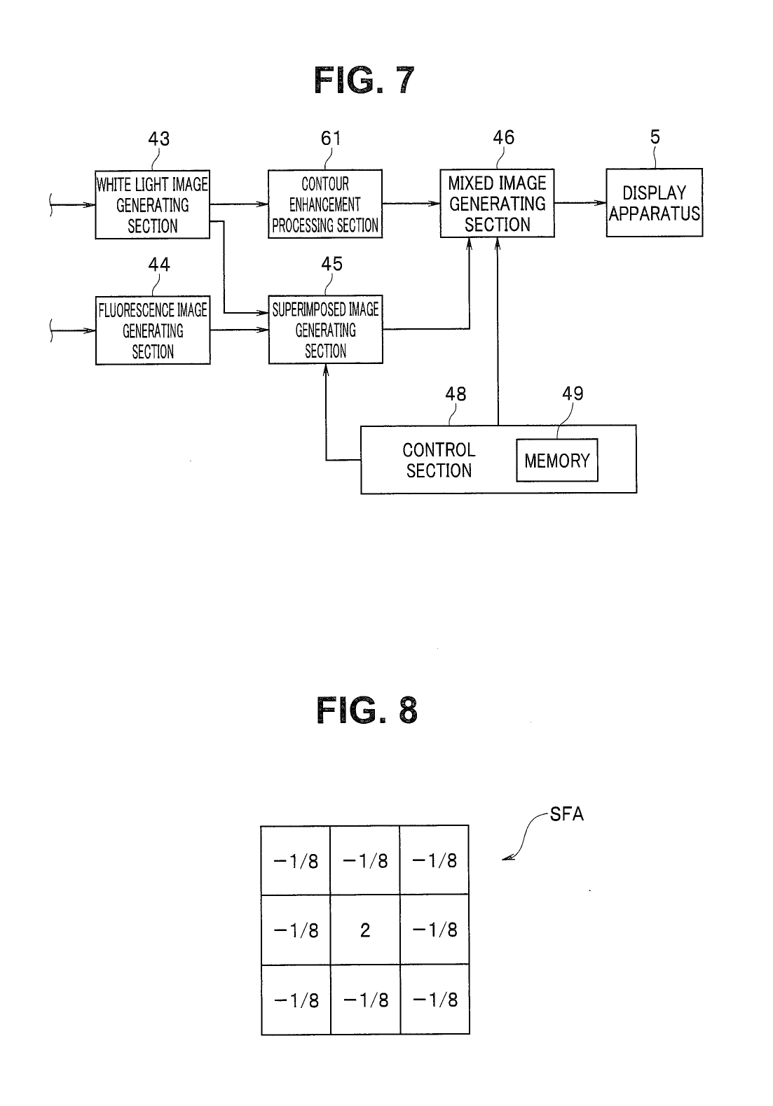

Also, according to the present embodiment, for example, as illustrated in FIG. 7, a contour enhancement processing section 61 configured to perform contour enhancement processing for enhancing a contour in a white light image WIA outputted from the white light image generating section 43 and output the white light image WIA subjected to the contour enhancement processing to the mixed image generating section 46 may further be provided (in the processor 4). In such a case, contour enhancement processing applying, for example, a spatial filter SFA, which is a sharpening filter of a size of 3×3 pixels such as illustrated in FIG. 8, to pixels WP included in a white light image WIA outputted from the white light image generating section 43 may be performed in the contour enhancement processing section 61. FIG. 7 is a diagram illustrating an example of a configuration where an observation image is generated using a white light image subjected to contour enhancement processing by a contour enhancement processing section. FIG. 8 is a diagram illustrating an example of a spatial filter that can be used in the contour enhancement processing by the contour enhancement processing section in FIG. 7.

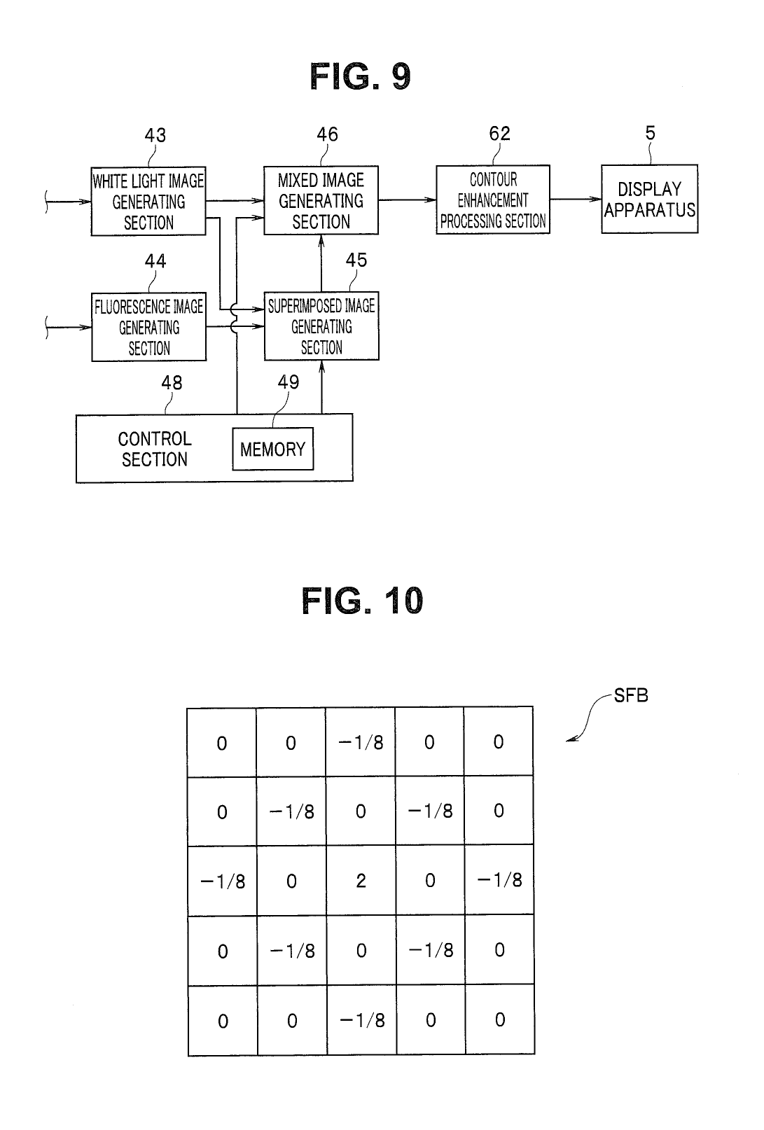

Also, according to the present embodiment, for example, as illustrated in FIG. 9, a contour enhancement processing section 62 configured to perform contour enhancement processing for enhancing a contour in an observation image outputted from the mixed image generating section 46 and output the observation image subjected to the contour enhancement processing to the display apparatus 5 may be further provided (in the processor 4). In such a case, for example, contour enhancement processing applying, for example, a spatial filter SFB, which is a sharpening filter of a size of 5×5 pixels such as illustrated in FIG. 10, to respective pixels WP in a pixel group WGA included in an observation image outputted from the mixed image generating section 46 may be performed in the contour enhancement processing section 62. However, it is desirable that the contour enhancement processing using the spatial filter SFB in FIG. 10 be performed for an observation image including a pixel group WGA extracted according to a checkered pixel array such as illustrated in FIG. 2. FIG. 9 is a diagram illustrating an example where an observation image is directly subjected to contour enhancement processing by a contour enhancement processing section. FIG. 10 is a diagram illustrating an example of a spatial filter that can be used in the contour enhancement processing by the contour enhancement processing section in FIG. 9.

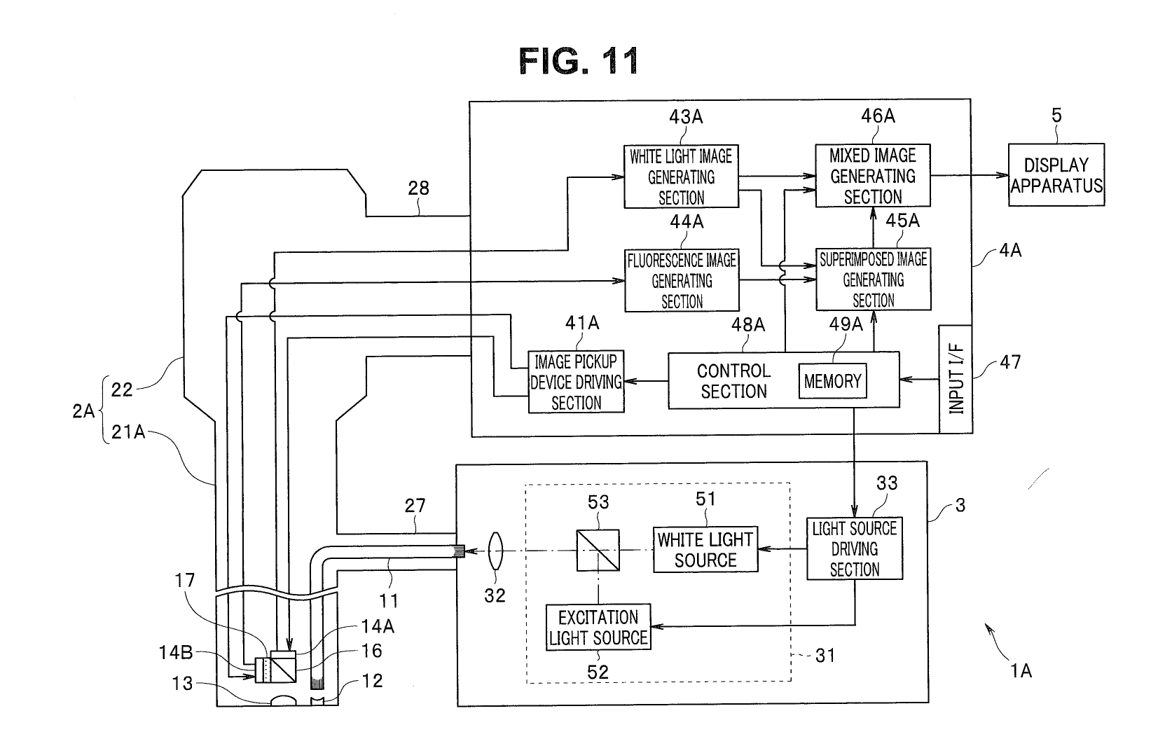

Also, the present embodiment is applicable not only to the endoscope system 1 illustrated in FIG. 1 but also to, for example, an endoscope system 1A, which is illustrated in FIG. 11, in a manner that is substantially similar to the above.

Here, a configuration of an endoscope system 1A according to a first modification of the present embodiment will be described. In the below, for simplicity, a detailed description of parts to which the already-described configurations or operation or the like can be applied will arbitrarily be omitted.

As illustrated in FIG. 11, an endoscope system 1A includes, for example, an endoscope 2A to be inserted into a body cavity of a subject, the endoscope 2A being configured to pick up an image of an object, such as a living body tissue, present inside the body cavity and output an image pickup signal, a light source apparatus 3 configured to supply light to be applied to the object to the endoscope 2A, a processor 4A configured to generate an observation image by performing various types of processing on the image pickup signal outputted from the endoscope 2A and output the observation image, and a display apparatus 5 configured to display the observation image outputted from the processor 4A on a screen. FIG. 11 is a diagram illustrating a configuration of a major part of an endoscope system according to a first modification of the embodiment.

As illustrated in FIG. 11, the endoscope 2A includes, for example, an insertion portion 21A formed in an elongated shape so that the insertion portion 21A can be inserted into a body cavity of a subject, and an operation section 22 provided on the proximal end side of the insertion portion 21A.

A light guide 11 for transmitting light supplied from the light source apparatus 3 is inserted inside the insertion portion 21A.

An illumination lens 12 and an objective lens 13 are provided in a distal end portion of the insertion portion 21A. Also, image pickup devices 14A and 14B, a spectral optical system 16 and an excitation light cut filter 17 are provided in the distal end portion of the insertion portion 21A.

The image pickup device 14A includes, for example, a color CMOS image sensor that includes a primary color filter or a complimentary color filter attached to an image pickup surface and is configured to perform image pickup operation in response to an image pickup device driving signal outputted from the processor 4A. Also, the image pickup device 14A is configured to pick up an image of light passed through the spectral optical system 16, generate an image pickup signal and output the generated image pickup signal to the processor 4A.

The image pickup device 14B includes, for example, a monochrome CMOS image sensor and is configured to perform image pickup operation in response to an image pickup device driving signal outputted from the processor 4A. Also, the image pickup device 14B is configured to pick up an image of light reflected by the spectral optical system 16 and passed through the excitation light cut filter 17, generate an image pickup signal and output the generated image pickup signal to the processor 4A.

The spectral optical system 16 includes, for example, a dichroic prism. Also, the spectral optical system 16 has an optical characteristic of transmitting light having a wavelength band of less than 700 nm in light entered via the objective lens 13 to the image pickup device 14A side and reflecting light having a wavelength band of no less than 700 nm in the light entered via the objective lens 13 to the excitation light cut filter 17 side.

The excitation light cut filter 17 is disposed on an optical path from the spectral optical system 16 to the image pickup device 14B. Also, the excitation light cut filter 17 has an optical characteristic of blocking a wavelength band that is the same as a wavelength band of excitation light EXA and transmitting wavelength bands that are different from the wavelength band of excitation light EXA, from among respective wavelength bands included in light reflected by the spectral optical system 16. In other words, the excitation light cut filter 17 has an optical characteristic of transmitting fluorescence FLA emitted from a fluorescent medical agent in response to application of excitation light EXA.

In other words, an image pickup section according to the present modification includes the image pickup devices 14A and 14B, the spectral optical system 16 and the excitation light cut filter 17.

As illustrated in FIG. 11, the processor 4A includes, for example, an image pickup device driving section 41A, a white light image generating section 43A, a fluorescence image generating section 44A, a superimposed image generating section 45A, a mixed image generating section 46A, an input I/F 47 and a control section 48A. Note that according to the present modification, for example, each of the sections of the processor 4A may be configured as an individual electronic circuit or may be configured as a circuit block in an integrated circuit such as an FPGA (field-programmable gate array).

The image pickup device driving section 41A is configured to generate an image pickup device driving signal for driving the image pickup devices 14A and 14B, based on a control signal outputted from the control section 48A and output the image pickup device driving signal.

The white light image generating section 43A is configured to generate a white light image WIA based on an image pickup signal outputted from the image pickup device 14A and output the generated white light image WIA to each of the superimposed image generating section 45A and the mixed image generating section 46A. In other words, the white light image generating section 43A is configured to generate a white light image WIA, which is an image obtained by performing image pickup of reflected light of white light WLA by the image pickup device 14A.

The fluorescence image generating section 44A is configured to generate a fluorescence image FIA based on an image pickup signal outputted from the image pickup device 14B and output the generated fluorescence image FIA to the superimposed image generating section 45A. In other words, the fluorescence image generating section 44A is configured to generate a fluorescence image FIA, which is an image obtained by performing image pickup of reflected light of fluorescence FLA by the image pickup device 14B.

The superimposed image generating section 45A is configured to be capable of performing operation according to a control signal outputted from the control section 48A. Also, the superimposed image generating section 45A is configured to generate a superimposed image SIA by performing processing for superimposing a white light image WIA outputted from the white light image generating section 43A and a fluorescence image FIA outputted from the fluorescence image generating section 44A on each other and output the generated superimposed image SIA to the mixed image generating section 46A. In other words, the superimposed image generating section 45A is configured to generate a superimposed image SIA, which is an image indicating a site of emission of fluorescence FLA in an object subjected to image pickup by the endoscope 2A.

The mixed image generating section 46A is configured to generate an observation image by performing processing for mixing a part of a white light image WIA outputted from the white light image generating section 43A and a part of a superimposed image SIA outputted from the superimposed image generating section 45A based on a control signal outputted from the control section 48A and output the generated observation image to the display apparatus 5.

The control section 48A is configured to be capable of generating control signals for causing operation according to an instruction from the input I/F 47 to be performed and outputting the control signals to the light source driving section 33, the superimposed image generating section 45A and the mixed image generating section 46A, respectively. Also, the control section 48A includes a memory 49A in which control information to be used for controlling respective sections of the endoscope system 1A is stored.

The control section 48A is configured to generate a control signal for extracting pixels WP included in a white light image WIA and pixels SP included in a superimposed image SIA according to a set of pixel arrays that do not overlap each other and each have periodicity, based on control information read from the memory 49A and output the control signal to the mixed image generating section 46A.

Here, in the present modification, instead of the light emitting section 31, for example, a broadband light source configured to emit broadband light that is light at least having a wavelength band in a range from a blue range to a near-infrared range, and an optical filter having an optical characteristic of transmitting wavelength bands that are the same as wavelength bands of white light WLA and excitation light EXA and blocking the other wavelength bands, from among respective wavelength bands included in the broadband light, may be provided in the light source apparatus 3.

Next, operation, etc., of the endoscope system 1A according to the present modification will be described.

First, after connecting the respective sections of the endoscope system 1A and turning on the power, a user provides, for example, an instruction for starting a fluorescence observation of an object to the control section 48A by operating a fluorescence observation start switch in the input I/F 47. Also, the user disposes the distal end portion of the insertion portion 21A in the vicinity of a desired object present inside a body cavity of a subject by inserting the insertion portion 21A into the body cavity.

When the processor 4A is powered up, the control section 48A reads control information from the memory 49A and generates a control signal for extracting pixels WP included in a white light image WIA and pixels SP included in a superimposed image SIA according to a set of pixel arrays that do not overlap each other and each have periodicity, based on the read control information and outputs the control signal to the mixed image generating section 46A.

When the processor 4A is powered up and the control section 48A detects the instruction from the fluorescence observation start switch in the input I/F 47, the control section 48A generates a control signal for causing the image pickup devices 14A and 14B to perform rolling shutter-type image pickup operation and outputs the control signal to the image pickup device driving section 41A, and generates a control signal for causing white light WLA in a light amount AL1 and excitation light EXA in the light amount AL1 to be generated simultaneously and outputs the control signal to the light source driving section 33.

Then, according to such control as above by the control section 48A, white light WLA and excitation light EXA are simultaneously applied to an object, an image of reflected light of white light WLA included in return light emitted from the object is picked up by the image pickup device 14A and an image of fluorescence FLA included in the return light is picked up by the image pickup device 14B. Also, according to such control as above by the control section 48A, an image pickup signal generated by the image pickup device 14A is outputted to the white light image generating section 43A, an image pickup signal generated by the image pickup device 14B is outputted to the fluorescence image generating section 44A, a white light image WIA generated by the white light image generating section 43A is outputted to the superimposed image generating section 45A and the mixed image generating section 46A, and a fluorescence image FIA generated by the fluorescence image generating section 44A is outputted to the superimposed image generating section 45A.

The superimposed image generating section 45A generates a superimposed image SIA by, for example, superimposing the white light image WIA outputted from the white light image generating section 43A and the fluorescence image FIA outputted from the fluorescence image generating section 44A on each other in a state in which coefficients α, β and γ in equation (1) above are set as α=γ=0 and β=1 and outputs the generated superimposed image SIA to the mixed image generating section 46A.

The mixed image generating section 46A generates an observation image including pixel arrays such as illustrated in FIG. 2 by performing processing for mixing a part of the white light image WIA outputted from the white light image generating section 43A and a part of the superimposed image SIA outputted from the superimposed image generating section 45A based on the control signal outputted from the control section 48A, and outputs the generated observation image to the display apparatus 5.

In other words, the endoscope system 1A according to the present modification enables generating an observation image that is similar to an observation image generated in the endoscope system 1 and displaying the observation image on the display apparatus 5. Therefore, the present modification enables when fluorescence emitted from a living body tissue is observed, easily grasping a structure of the living body tissue at a site of emission of the fluorescence in comparison with the conventional techniques.

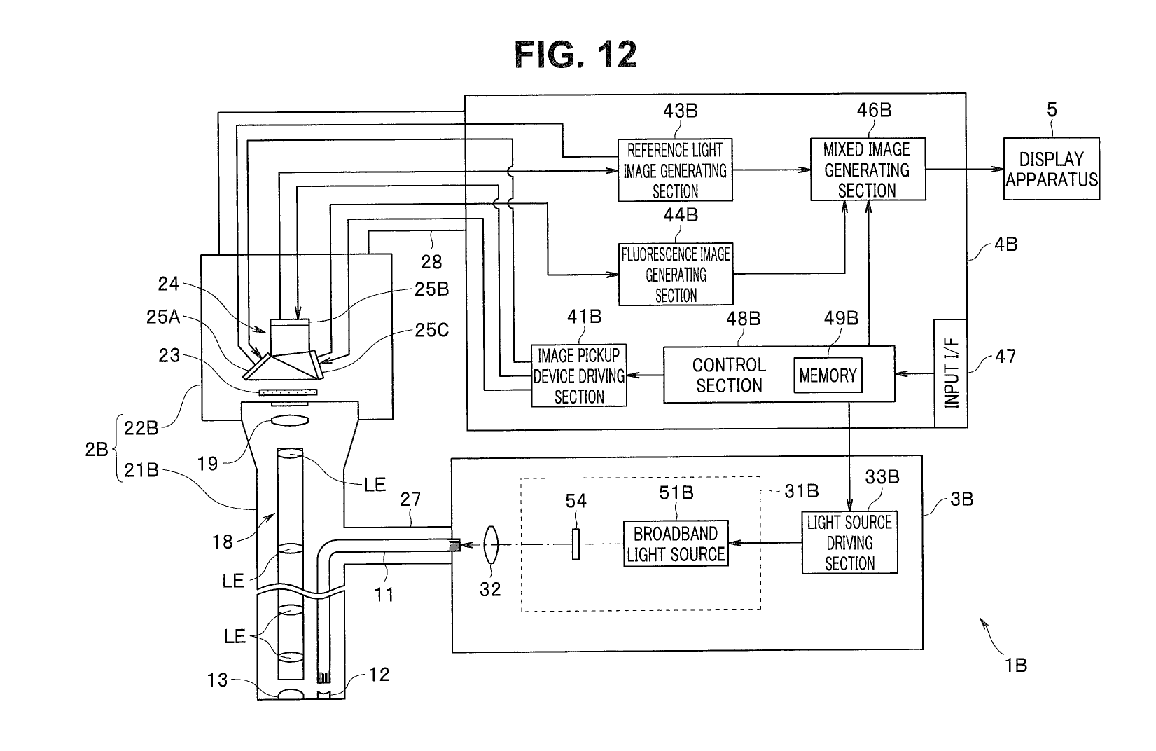

Also, the present embodiment is applicable not only to the endoscope system 1 illustrated in FIG. 1 but also to, for example, an endoscope system 1B, which is illustrated in FIG. 12, in a manner that is substantially similar to the above.

Here, a configuration of an endoscope system 1B according to a second modification of the present embodiment will be described.

As illustrated in FIG. 12, the endoscope system 1B includes, for example, an endoscope 2B to be inserted into a body cavity of a subject, the endoscope 2B being configured to pick up an image of an object, such as a living body tissue, present inside the body cavity and output an image pickup signal, a light source apparatus 3B configured to supply light to be applied to the object to the endoscope 2B, a processor 4B configured to generate an observation image by performing various types of processing on the image pickup signal outputted from the endoscope 2B and output the observation image, and a display apparatus 5 configured to display the observation image outputted from the processor 4B on a screen. FIG. 12 is a diagram illustrating a configuration of a major part of an endoscope system according to a second modification of the embodiment.

The endoscope 2B includes an optical viewing tube 21B having an elongated shape, and a camera unit 22B that is detachably attachable to a proximal end portion of an optical viewing tube 21B.

The optical viewing tube 21B has a function as an insertion portion that can be inserted into a body cavity of a subject.

A light guide 11 for transmitting light supplied from the light source apparatus 3B is inserted inside the optical viewing tube 21B.

An illumination lens 12 and an objective lens 13 are provided in a distal end portion of the optical viewing tube 21B.

As illustrated in FIG. 12, a relay lens 18 including a plurality of lenses LE for transmitting light entered from the objective lens 13 is provided inside the optical viewing tube 21B. In other words, the relay lens 18 has a function as a transmission optical system configured to transmit light entered from the objective lens 13.

As illustrated in FIG. 12, an eyepiece 19 for enabling an optical image of an object according to light transmitted from the relay lens 18 to be observed by the naked eye is provided in the proximal end portion of the optical viewing tube 21B.

The camera unit 22B has a function as an image pickup section and includes an excitation light cut filter 23, a dichroic prism 24 and image pickup devices 25A, 25B and 25C.

The excitation light cut filter 23 is disposed on a front face of the dichroic prism 24 and has an optical characteristic of blocking a wavelength band that is the same as a wavelength band of excitation light EXA and transmitting wavelength bands that are different from the wavelength band of excitation light EXA, from among respective wavelength bands included in light outputted via the eyepiece 19. In other words, the excitation light cut filter 23 has an optical characteristic of transmitting fluorescence FLA emitted from a fluorescent medical agent in response to application of excitation light EXA.

The dichroic prism 24 is configured to split light outputted via the excitation light cut filter 23 into three wavelength bands, light in a range from a red range to a near-infrared range, light in a green range and light in a blue range.

The image pickup device 25A includes, for example, a monochrome CMOS image sensor and is configured to perform image pickup operation in response to an image pickup device driving signal outputted from the processor 4B. Also, the image pickup device 25A is configured to pick up an image of blue range light outputted via the dichroic prism 24, generate an image pickup signal according to the picked-up image of blue range light and output the image pickup signal.

The image pickup device 25B includes, for example, a monochrome CMOS image sensor and is configured to perform image pickup operation in response to an image pickup device driving signal outputted from the processor 4B. Also, the image pickup device 25B is configured to pick up an image of green range light outputted via the dichroic prism 24, generate an image pickup signal according to the picked-up image of the green range light and output the image pickup signal.

The image pickup device 25C includes, for example, a monochrome CMOS image sensor and is configured to perform image pickup operation in response to an image pickup device driving signal outputted from the processor 4B. Also, the image pickup device 25C is configured to pick up an image of light in the range from the red range to the near-infrared range, the light being outputted via the dichroic prism 24, generate an image pickup signal according to the picked-up image of the light in the range from the red range to the near-infrared range and output the image pickup signal.

As illustrated in FIG. 12, the light source apparatus 3B includes, for example, a light emitting section 31B, a collective lens 32 and a light source driving section 33B.

The light emitting section 31B includes a broadband light source 51B and an optical filter 54.

The broadband light source 51B includes, for example, a lamp configured to emit broadband light that is light at least having a wavelength band in a range from the blue range to the near-infrared range. Also, the broadband light source 51B is configured to emit broadband light in response to a driving signal outputted from the light source driving section 33B.

The optical filter 54 has an optical characteristic of transmitting a wavelength band that is the same as a wavelength band of excitation light EXA and transmitting the blue range and the green range at a transmittance of around 10%, from among respective wavelength bands of broadband light emitted from the broadband light source 51B. Also, the optical filter 54 has an optical characteristic of blocking a wavelength band other than the blue range, the green range and the wavelength band of excitation light EXA from among respective wavelength bands included in broadband light outputted from the broadband light source 51B.

In other words, the light emitting section 31B is configured to be capable of simultaneously emitting reference light RLA, which is light having two wavelength bands in the blue range and the green range, and excitation light EXA by causing the broadband light source 51B to emit light in response to a driving signal outputted from the light source driving section 33B. Also, the light emitting section 31B is configured to be capable of outputting reference light RLA and excitation light EXA to the collective lens 32.

Note that the light emitting section 31B according to the present modification may include, for example, a blue LED configured to emit blue light, a green LED configured to emit green light and an LD configured to emit excitation light EXA. Alternatively, the light emitting section 31B according to the present modification may include, for example, a white LED configured to emit white light WLA, an optical filter having an optical characteristic of transmitting only a wavelength band that is the same as a wavelength of reference light RLA from among respective wavelength bands included in the white light WLA, and an LD configured to emit excitation light EXA. Alternatively, the light emitting section 31B according to the present modification may include a broadband light source 51B, an optical filter having an optical characteristic of transmitting the blue range and the green range at a transmittance of around 10% and blocking the other wavelength bands, from among respective wavelength bands included in broadband light emitted from the broadband light source 51B, and an LD configured to emit excitation light EXA.

The light source driving section 33B is configured to generate a light source driving signal for driving the broadband light source 51B, based on a control signal outputted from the processor 4B and output the light source driving signal to the light emitting section 31B.

In other words, the light source apparatus 3B is configured to be capable of emitting excitation light EXA for exciting a fluorescent medical agent administered to a subject and reference light RLA, which is illuminating light for illuminating the inside of a body cavity of the subject.

As illustrated in FIG. 12, the processor 4B includes, for example, an image pickup device driving section 41B, a reference light image generating section 43B, a fluorescence image generating section 44B, a mixed image generating section 46B, an input I/F 47 and a control section 48B. Note that according to the present modification, for example, each of the sections of the processor 4B may be configured as an individual electronic circuit or may be configured as a circuit block in an integrated circuit such as an FPGA (field-programmable gate array).

The image pickup device driving section 41B is configured to generate an image pickup device driving signal for driving the image pickup devices 25A, 25B and 25C, based on a control signal outputted from the control section 48B and output the image pickup device driving signal.

The reference light image generating section 43B is configured to generate a reference light image RIA based on image pickup signals outputted from the image pickup devices 25A and 25B and output the generated reference light image RIA to the mixed image generating section 46B. In other words, the reference light image generating section 43B is configured to generate a reference light image RIA, which is an image obtained by performing image pickup of reflected light of reference light RLA by the image pickup devices 25A and 25B. Also, a reference light image RIA is generated as a cyan image obtained by combining a blue image obtained according to an image pickup signal outputted from the image pickup device 25A and a green image obtained according to an image pickup signal outputted from the image pickup device 25B.

The fluorescence image generating section 44B is configured to generate a fluorescence image FIA based on an image pickup signal outputted from the image pickup device 25C and output the generated fluorescence image FIA to the mixed image generating section 46B. Here, it is assumed that a fluorescence image FIA generated by the fluorescence image generating section 44B is generated as an image indicating a site of emission of fluorescence FLA in an object subjected to image pickup by the endoscope 2B in a color that is different from a color of a reference light image RIA. In other words, the fluorescence image generating section 44B is configured to generate a fluorescence image FIA, which is an image indicating a site of emission of fluorescence FLA in an object subjected to image pickup by the endoscope 2B.

The mixed image generating section 46B is configured to generate an observation image by performing processing for mixing a part of a reference light image RIA outputted from the reference light image generating section 43B and a part of a fluorescence image FIA outputted from the fluorescence image generating section 44B, based on a control signal outputted from the control section 48B and output the generated observation image to the display apparatus 5.

The control section 48B is configured to be capable of generating control signals for causing operation according to an instruction from the input I/F 47 to be performed and outputting the control signals to the light source driving section 33B and the mixed image generating section 46B, respectively. Also, the control section 48B includes a memory 49B in which control information to be used for controlling respective sections of the endoscope system 1B.

The control section 48B is configured to generate a control signal for extracting pixels RP included in a reference light image RIA and pixels FP included in a fluorescence image FIA according to a set of pixel arrays that do not overlap each other and each have periodicity, based on control information read from the memory 49B and output the control signal to the mixed image generating section 46B.

Next, operation, etc., of the endoscope system 1B according to the present modification will be described.

First, after connecting the respective sections of the endoscope system 1B and turning on the power, a user provides, for example, an instruction for starting a fluorescence observation of an object to the control section 48B by operating a fluorescence observation start switch in the input I/F 47. Also, the user disposes the distal end portion of the optical viewing tube 21B in the vicinity of a desired object present inside a body cavity of a subject by inserting the optical viewing tube 21B into the body cavity.

When the processor 4B is powered up, the control section 48B reads control information from the memory 49B and generates a control signal for extracting pixels RP included in a reference light image RIA and pixels FP included in a fluorescence image FIA according to a set of pixel arrays that do not overlap each other and each have periodicity, based on the read control information and outputs the control signal to the mixed image generating section 46B.

More specifically, for example, the control section 48B generates a control signal for extracting a pixel group RGA including a plurality of pixels RP present at positions in a first pixel array of the set of pixel arrays that do not overlap each other and each have periodicity, from among the respective pixels included in the reference light image RIA, and extracting a pixel group FGA including a plurality of pixels FP present at positions in a second pixel array of the set of pixel arrays, from among the respective pixels included in the fluorescence image FIA, and outputs the control signal to the mixed image generating section 46B.

When the processor 4B is powered up and the control section 48B detects the instruction from the fluorescence observation start switch in the input I/F 47, the control section 48B generates a control signal for causing the image pickup devices 25A, 25B and 25C to perform rolling shutter-type image pickup operation and outputs the control signal to the image pickup device driving section 41B and generates a control signal for causing emission of broadband light in a light amount AL1 and outputs the control signal to the light source driving section 33B.

According to such control as above by the control section 48B, reference light RLA and excitation light EXA are simultaneously applied to an object, an image of reflected light of the reference light RLA, the reflected light being included in return light emitted from the object, is picked up by the image pickup devices 25A and 25B, and an image of fluorescence FLA included in the return light is picked up by the image pickup device 25C. Also, according to such control as above by the control section 48B, image pickup signals generated by the image pickup devices 25A and 25B are outputted to the reference light image generating section 43B and an image pickup signal generated by the image pickup device 25C is outputted to the fluorescence image generating section 44B, and a reference light image RIA generated by the reference light image generating section 43B and a fluorescence image FIA generated by the fluorescence image generating section 44B are outputted respectively to the mixed image generating section 46B.

The mixed image generating section 46B generates an observation image by performing processing for mixing a part of the reference light image RIA outputted from the reference light image generating section 43B and a part of the fluorescence image FIA outputted from the fluorescence image generating section 44B based on the control signal outputted from the control section 48B, and outputs the generated observation image to the display apparatus 5.

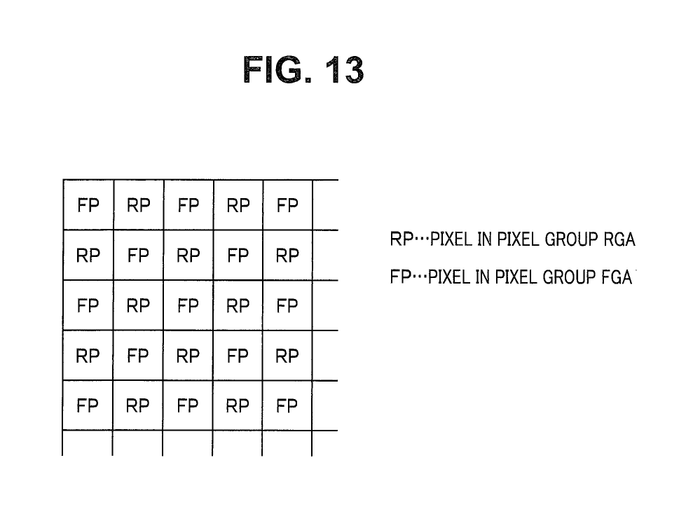

More specifically, the mixed image generating section 46B generates an observation image by, for example, extracting the pixel group RGA according to a first checkered pixel array and extracting the pixel group FGA according to a second checkered pixel array that does not overlap the first checkered pixel array, based on the control signal outputted from the control section 48B and mixing the extracted pixel groups RGA and FGA, and outputs the generated observation image to the display apparatus 5. As a result of such operation as above being performed by the mixed image generating section 46B, for example, as illustrated in FIG. 13, an observation image in which pixels RP and pixels FP are alternately disposed in a one pixel-by-one pixel basis and the number of pixels RP and the number of pixels FP are equal to each other is generated. Also, where such operation as above is performed in the mixed image generating section 46B, information for extracting the pixel group RGA according to the first checkered pixel array and extracting the pixel group FGA according to the second checkered pixel array that does not overlap the first checkered pixel array is included in the control information stored in the memory 49B. FIG. 13 is a diagram illustrating an example of pixel arrays in an observation image generated by an endoscope system of the second modification of the embodiment.

As described above, the endoscope system 1B according to the present modification enables displaying an observation image in which information indicating a site of emission of fluorescence FLA included in a fluorescence image FIA is added while information indicating a structure, including, e.g., projections and recesses, of a living body tissue included in a reference light image RIA is left as much as possible, on the display apparatus 5. Therefore, the present modification enables when fluorescence emitted from a living body tissue is observed, easily grasping a structure of the living body tissue at a site of emission of the fluorescence in comparison with the conventional techniques.

Note that the configuration of the camera unit 22B, etc., in the endoscope system 1B according to the present modification may arbitrarily be modified so that, for example, images of reflected light of white light WLA and fluorescence FLA included in return light emitted from an object to which white light WLA and excitation light EXA are applied in a time-division manner are picked up by a single image pickup device, a white light image WIA and a superimposed image SIA are generated based on image pickup signals outputted from the single image pickup device and an observation image in which the white light image WIA and the superimposed image SIA are mixed is generated. Also, the configuration of the camera unit 22B, etc., in the endoscope system 1B according to the present modification may arbitrarily be modified so that, for example, images of reflected light of white light WLA and fluorescence FLA included in return light emitted from an object to which white light WLA and excitation light EXA are applied simultaneously are picked up by two image pickup devices, and a white light image WIA and a superimposed image SIA are generated based on image pickup signals outputted from the two image pickup devices, and an observation image in which the white light image WIA and the superimposed image SIA are mixed is generated.

It should be understood that: the present invention is not limited to the respective embodiments and respective modifications described above; and various changes and applications are possible without departing from the spirit of the invention.

What is claimed is:

1. An endoscope system comprising:
a light source apparatus configured to be capable of emitting excitation light for exciting a fluorescent medical agent administered to a subject and illuminating light for illuminating an inside of a body cavity of the subject;
an image sensor configured to pick up an image of each of fluorescence emitted in response to application of the excitation light to an object present inside the body cavity of the subject to which the fluorescent medical agent is administered and reflected light emitted in response to application of the illuminating light to the object; and
a mixed image generating circuit configured to generate an observation image in which a first image that is an image obtained by performing image pickup of the reflected light by the image sensor, and a second image that is an image indicating a site of emission of the fluorescence in the object are mixed in a set of pixel arrays that do not overlap each other and each have periodicity.

2. The endoscope system according to claim 1, further comprising a control circuit configured to generate a control signal for extracting a first pixel group from respective pixels included in the first image and extracting a second pixel group from respective pixels included in the second image, according to the set of pixel arrays that do not overlap each other and each have periodicity and output the control signal to the mixed image generating section.

3. The endoscope system according to claim 2, wherein the control circuit causes the first pixel group to be extracted according to a first checkered pixel array of the set of pixel arrays and causes the second pixel group to be extracted according to a second checkered pixel array of the set of pixel arrays.

4. The endoscope system according to claim 2, wherein the control circuit causes the first pixel group to be extracted according to a first vertically striped pixel array of the set of pixel arrays and causes the second pixel group to be extracted according to a second vertically striped pixel array of the set of pixel arrays.

5. The endoscope system according to claim 2, wherein the control circuit causes the first pixel group to be extracted according to a first horizontally striped pixel array of the set of pixel arrays and causes the second pixel group to be extracted according to a second horizontally striped pixel array of the set of pixel arrays.

6. The endoscope system according to claim 2, wherein:
the control circuit generates a control signal for extracting a pixel present at a predetermined position in each of small areas of 2×2 pixels obtained as a result of the first image being divided into the small areas, as a pixel belonging to the first pixel group and extracting respective pixels present at positions other than the predetermined position in each of small areas of 2×2 pixels obtained as a result of the second image being divided into the small areas, as pixels belonging to the second pixel group; and
the endoscope system further includes an input interface enabling providing an instruction for changing a ratio between the first pixel group and the second pixel group to the control circuit.

7. The endoscope system according to claim 1, further comprising a superimposed image generating circuit configured to generate a superimposed image in which the first image and a fluorescence image that is an image obtained by performing image pickup of the fluorescence by the image sensor are superimposed on each other, as the second image.

8. The endoscope system according to claim 7, wherein the superimposed image generating circuit and the mixed image generating circuit are configured integrally.

9. The endoscope system according to claim 7, wherein:
the control circuit generates a control signal for extracting a pixel present at a predetermined position in each of small areas of 2×2 pixels obtained as a result of the first image being divided into the small areas, as a pixel belonging to the first pixel group and extracting respective pixels present at positions other than the predetermined position in each of small areas of 2×2 pixels obtained as a result of the superimposed image being divided into the small areas, as pixels belonging to the second pixel group; and
when the superimposed image generating circuit generates the superimposed image, the superimposed image generating circuit sets a weight coefficient applied to the fluorescence image, based on the control signal from the control circuit.

10. The endoscope system according to claim 2, wherein the light source apparatus sets a light amount for the illuminating light based on a control signal from the control circuit.

11. The endoscope system according to claim 6, wherein the input interface is configured to enable setting a type of surgical operation performed for the subject and/or a type of the fluorescent medical agent.

12. The endoscope system according to claim 1, further comprising a contour enhancement processing circuit configured to perform contour enhancement processing for enhancing a contour in the first image and output the first image subjected to the contour enhancement processing to the mixed image generating circuit.

13. The endoscope system according to claim 1, further comprising a contour enhancement processing circuit configured to perform contour enhancement processing for enhancing a contour in the observation image generated by the mixed image generating circuit.
